(19) United States
(12) Patent Application Publication
(10) Pub. No.: US 2026/0083949 A1
(43) Pub. Date: Mar. 26, 2026

(54) MICRONEEDLE PATCH AND METHOD FOR MANUFACTURING THE SAME

(71) Applicants: Gachon University of Industry-Academic Cooperation Foundation, Seongnam-si (KR); QuadMedicine, Inc., Seongnam-si (KR)

(72) Inventors: Seung Ki BAEK, Seoul (KR); Jung Hwan PARK, Seongnam-si (KR); Min ju KWON, Seongnam-si (KR); Min Woo RYU, Yongin-si (KR)

(73) Assignees: Gachon University of Industry-Academic Cooperation Foundation, Seongnam-si (KR); QuadMedicine, Inc., Seongnam-si (KR)

(21) Appl. No.: 19/336,809

(22) Filed: Sep. 23, 2025

(30) Foreign Application Priority Data
Sep. 24, 2024 (KR) 10-2024-0128629
Sep. 17, 2025 (KR) 10-2025-0133316

(51) Int. Cl. A61M 37/00

(52) U.S. Cl. CPC A61M 37/0015; A61M 2037/0023; A61M 2037/0053; A61M 2205/0238

(57) ABSTRACT

Disclosed is a microneedle patch, including: a microneedle part including a base and a plurality of needles for forming tips protruding in a sharp shape from one surface of the base; and a polymer fiber layer formed to cover the microneedle part to perform a function of protecting an active pharmaceutical ingredient contained in the needles from moisture and contaminants, wherein the edge of the polymer fiber layer is connected to the edge of one surface of the base, and a central part of the polymer fiber layer is supported in a state of being stacked and placed on tips of the needles, and the edge of the polymer fiber layer may be connected to the edge of one surface of the base, and the central part of the polymer fiber layer may be supported in a state of being stacked and placed on the tips of the needles.

### BACKGROUND OF THE DISCLOSURE

### Field of the Disclosure

The present disclosure relates to a microneedle patch having a drug-protecting layer formed thereon and a method of manufacturing the same. More specifically, the present disclosure relates to a technology for protecting microneedles from moisture and external contaminants by using a fiber layer formed by an electrospinning method.

### Description of the Related Art

A microneedle is a technology that can deliver drugs (pharmaceutical ingredients) to the epidermis and dermis layers in a minimally invasive manner, and its application range is expanding to preventive vaccines, therapeutics, and cosmetics due to less pain and high user convenience.

In particular, the demand and applications for microneedles are steadily increasing due to the increase in the elderly population, the expansion of the self-administration market, and the increased preference for non-invasive and low-pain drug delivery technologies. Such market needs are promoting the development of various microneedle products, such as solid, coated, and dissolvable formulations.

However, microneedles developed so far may experience a decrease in mechanical strength, shape deformation, component loss, and efficacy degradation when exposed to external environmental contaminants such as moisture, fine particles, and bacteria, due to the physical and chemical properties of the formulation's constituent materials.

These environmental effects may impair skin penetration or reduce drug delivery efficiency, and act as a major factor in deteriorating the quality and stability of the product during the entire process including storage, transportation, and administration. Particularly in high-temperature, high-humidity environments or environments where hygiene management is difficult, there is a problem that the drug permeation rate or drug performance is lowered below a certain level due to contaminants.

To date, simple physical protection devices such as plastic cases or blister packs have been used for the protection of microneedle formulations. However, this method has a limitation in that it cannot prevent drug denaturation due to contaminants even during the short time from when the package is removed until it is inserted into the skin. In addition, when sweat or moisture is present on the skin, the solid formulation at the tip part may be partially dissolved by the moisture during the administration of the microneedles, which may cause the drug delivery efficiency to fluctuate.

To compensate for this, various methods such as barrier films and sealing coatings have been proposed, but most have the problem of deteriorating the insertability or having limited application depending on the formulation of the microneedles.

Therefore, there is a need for a protective technology that can be integrally applied regardless of the microneedle formulation and does not affect the moisture barrier and external contaminant barrier functions, as well as the insertability.

### RELATED ART DOCUMENT

### Non-Patent Document

(Non-patent Document 001) Scientific Reports. (2024) 14:19228, “Microneedle patch casting using a micromachined carbon master for enhanced drug delivery”

### SUMMARY OF THE DISCLOSURE

Therefore, the present disclosure has been made in view of the above problems, and it is an object of the present disclosure to provide a protective layer that can protect microneedles from contaminants such as moisture and pollutants during long-term storage and exposure of the microneedles.

It is another object of the present disclosure to design a protective layer that does not deteriorate the skin insertability of microneedles or inhibit their drug delivery function even if the microneedles have a protective layer.

It is yet another object of the present disclosure to design a protective layer that can be applied to various formulations of microneedles, such as coated, solid, and dissolvable formulations.

In accordance with an aspect of the present disclosure, the above and other objects can be accomplished by the provision of a microneedle patch, including: a microneedle part including a base and a plurality of needles for forming tips protruding in a sharp shape from one surface of the base; and a polymer fiber layer formed to cover the microneedle part to perform a function of protecting an active pharmaceutical ingredient contained in the needles from moisture and contaminants.

Here, an edge of the polymer fiber layer may be connected to the edge of one surface of the base, and a central part of the polymer fiber layer may be supported in a state of being stacked and placed on tips of the needles.

Preferably, the needles may be made of at least one of a solid formulation, a dissolvable formulation, a powder-attached formulation, and a coated formulation. The tips of the needles may pass through the polymer fiber layer and may be inserted into skin when the microneedle patch is used.

Preferably, the polymer fiber layer may include a lipophilic and biocompatible polymer, and form a stacked structure in a form of a porous mesh or film including pores.

The polymer fiber layer may further include a hygroscopic material to suppress denaturation of the active pharmaceutical ingredient and denaturation of the needles due to moisture absorption during long-term storage.

The polymer fiber layer may increase hydrophobicity by a porous fiber structure including the pores and by surface roughness to suppress penetration of external moisture.

The polymer fiber layer may control the size of the pores and an electrostatic interaction between the fibers and the contaminants to suppress the contaminants from reaching a surface of the microneedle part.

The tips of the needles may pass through the pores, formed in the mesh-form polymer fiber layer, and may be inserted into skin, when the microneedle part is used.

The tips of the needles may pass through the film-form polymer fiber layer by locally fracturing the film-form polymer fiber layer due to high local stress acting on the film-form polymer fiber layer and may be inserted into skin, when the microneedle part is used.

Preferably, the polymer fiber layer may have a thickness of 10 to 50 μm.

Preferably, the polymer fiber layer may have a pore size of 0.5 to 4 μm.

In accordance with another aspect of the present disclosure, provided is a method of manufacturing a microneedle patch, the method including: forming a microneedle part including a base and a plurality of needles for forming tips protruding in a sharp shape from one surface of the base; and forming a polymer fiber layer formed to cover the microneedle part by electrospinning a lipophilic and biocompatible polymer solution onto the microneedle part to perform a function of protecting an active pharmaceutical ingredient contained in the needles from moisture and contaminants.

Here, the edge of the polymer fiber layer may be connected to the edge of one surface of the base, and the central part of the polymer fiber layer may be supported in a state of being stacked and placed on tips of the needles.

Preferably, in the forming of the polymer fiber layer, the polymer solution may include polycaprolactone (PCL).

Preferably, in the forming of the polymer fiber layer, the polymer solution may include a cosolvent to ensure shape stability and fiber uniformity of the polymer fiber layer.

The cosolvent may include acetone and acetic acid. Here, a composition ratio of the acetone to the acetic acid may be 7:3 to 9:1.

Preferably, in the forming of the polymer fiber layer, the polymer solution may be electrospun at an electrospinning speed of 8 to 10 mL/h.

### BRIEF DESCRIPTION OF THE DRAWINGS

The above and other objects, features and other advantages of the present disclosure will be more clearly understood from the following detailed description taken in conjunction with the accompanying drawings, in which:

FIGS. 1A and 1B illustrate the schematic views of a microneedle patch according to an embodiment of the present disclosure.

FIGS. 2A and 2B illustrate the images of PCL fibers electrospun according to the method of the preparation example, observed with a scanning electron microscope (SEM);

FIG. 3 illustrates the results of a quantitative analysis of the pore size distribution of the electrospun PCL fiber layer observed in FIGS. 2A and 2B;

FIG. 4 illustrates the results comparing the surface contact angle of PCL fibers electrospun according to the preparation example with that of a solid film prepared from a PCL solution of the same composition;

FIG. 5 is a set of stress-strain curves illustrating the mechanical properties of PCL fibers of various thicknesses prepared by controlling the spinning amount of a polycaprolactone (PCL) solution;

FIGS. 6A to 6C illustrate the schematic view of an electrospinning process dependent upon the presence or absence of a conductive frame and the microneedle part;

FIGS. 7A and 7D are a set of images illustrating the structure of an electrospun fiber layer formed on the top of the microneedle part according to an embodiment of the present disclosure and the insertability of the microneedle part;

FIGS. 8A and 8B illustrate a set of optical microscope images of a polycaprolactone (PCL) mesh formed on the top of a microneedle part, dependent upon the presence or absence of a conductive frame;

FIGS. 9A to 9F are a set of optical microscope images illustrating a difference in the penetration heights of tips of a microneedle part, exposed when it penetrates a skin simulant (polydimethylsiloxane, PDMS), dependent upon a change in the thickness of a PCL mesh formed through an electrospinning process;

FIG. 10 is a graph illustrating the results of measuring the insertion depths of a microneedle part without a protective layer (bare needles) and a microneedle part with a protective layer (E-MAP) through in-vitro insertion experiments using Parafilm M®;

FIGS. 11A to 11D illustrate the results of evaluating the moisture protection ability of a PCL mesh protective layer for a microneedle part coated with trypan blue;

FIGS. 12A to 12D illustrate observation results obtained using confocal laser scanning microscopy to evaluate whether a microneedle part-coating layer is damaged by water spraying;

FIG. 13 illustrates a change in a contact angle dependent upon the time a PCL mesh is exposed to moisture, and FIGS. 14A to 14D illustrate optical microscope images of a PCL mesh and microneedle part-coating layer dependent upon the time of exposure to moisture;

FIGS. 15A to 15D illustrate optical microscope images of contaminant particles of various sizes, the inset in each image is an SEM image for confirming the average diameter of the contaminant particles, and FIGS. 16A to 16D illustrate SEM images for observing the adhesion pattern of contaminant particles of various sizes on the PCL mesh;

FIGS. 17A to 17D illustrate the surface images of microneedle parts observed with an optical microscope after removing the PCL mesh following the induction of penetration by contaminants of various sizes;

FIGS. 18A to 18C through FIGS. 20A to 20C illustrate the optical microscope images of bare needles (FIGS. 18A to 18C), E-C-MAP (FIGS. 19A to 19C), and C-MAP (FIGS. 20A to 20C) before and after permeating PDMS and pig skin; and

FIGS. 21A to 21C illustrates the confocal microscope images of the microneedle part before and after insertion into pig skin.

### DETAILED DESCRIPTION OF THE DISCLOSURE

The present disclosure will now be described more fully with reference to the accompanying drawings and contents disclosed in the drawings. However, the present disclosure should not be construed as limited to the exemplary embodiments described herein.

The terms used in the present specification are used to explain a specific exemplary embodiment and not to limit the present inventive concept. Thus, the expression of singularity in the present specification includes the expression of plurality unless clearly specified otherwise in context. It will be further understood that the terms “comprise” and/or “comprising”, when used in this specification, specify the presence of stated components, steps, operations, and/or elements, but do not preclude the presence or addition of one or more other components, steps, operations, and/or elements thereof.

It should not be understood that arbitrary aspects or designs disclosed in “embodiments”, “examples”, “aspects”, etc. used in the specification are more satisfactory or advantageous than other aspects or designs.

In addition, the expression “or” means “inclusive or” rather than “exclusive or”. That is, unless otherwise mentioned or clearly inferred from context, the expression “x uses a or b” means any one of natural inclusive permutations.

In addition, as used in the description of the disclosure and the appended claims, the singular forms “a”, “an” and “the” are intended to include the plural forms as well, unless context clearly indicates otherwise.

In addition, when an element such as a layer, a film, a region, and a constituent is referred to as being “on” another element, the element can be directly on another element or an intervening element can be present.

Hereinafter, embodiments of the present disclosure will be described in detail with reference to the accompanying drawings.

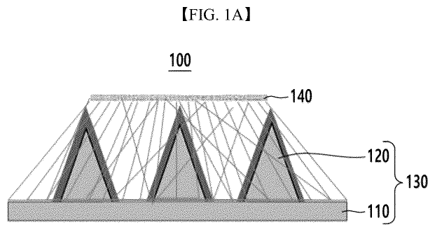

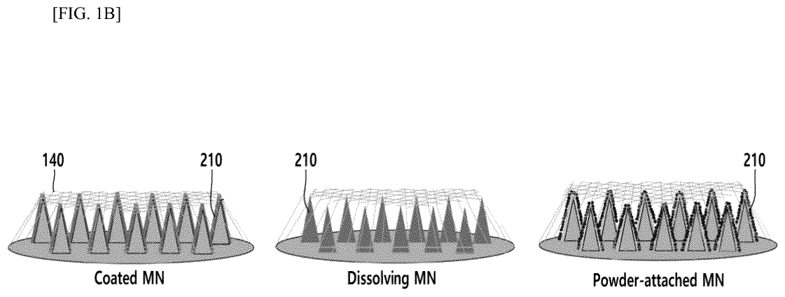

FIGS. 1A and 1B are schematic views of a microneedle patch according to an embodiment of the present disclosure.

As shown in FIG. 1A, a microneedle patch 100 according to an embodiment of the present disclosure may include a microneedle part 130 including a base 110 and a plurality of needles 120 for forming tips protruding in a sharp shape from one surface of the base 110, and a polymer fiber layer 140 formed to cover the microneedle part 130.

The microneedle part 130 is a structure that non-invasively penetrates the skin barrier to enable the delivery of pharmaceutical ingredients, vaccines, or physiologically active substances.

The base 110 may be formed in a planar or curved shape. The base 110 may support the plural needles 120 that form the tips of the microneedle part 130. Therefore, the base 110 may ensure the overall mechanical stability of the microneedle patch 100, and at the same time, may perform the role of uniformly distributing pressure upon contact with the skin.

The plural needles 120 may be formed in a shape extending from one surface of the base 110 into a sharp shape and protruding sharply. Therefore, the plural needles 120 may effectively penetrate the stratum corneum of the skin. The plural needles 120 may be adjusted in length in the range of tens to hundreds of micrometers (μm) to minimize pain and avoid reaching nerve and vascular tissues below the dermis layer. The needles 120 may be formed in a sharp shape such as a cone, a pyramid, or a cylinder. The tips of the needles 120 may be formed sharply to improve skin insertability.

In addition, as shown in FIGS. 1A and 1B, the microneedle part 130 of the microneedle patch 100 according to an embodiment of the present disclosure may be formed of at least one of a solid formulation, a dissolvable formulation, a powder-attached formulation, and a coated formulation.

The solid type formulation requires high mechanical strength and biocompatibility, and is a structural support mainly for the purpose of physical stimulation or skin penetration. The solid formulation may be formed using materials such as silicon, metal, or ceramic.

The dissolvable type formulation may be formed using biocompatible polymers such as hyaluronic acid, carboxymethyl cellulose, and polyvinylpyrrolidone. In the dissolvable formulation, an active pharmaceutical ingredient 210 loaded therein may be released as it dissolves upon contact with body fluids.

The powder-attached formulation may be formed in a structure in which particles of the active pharmaceutical ingredient 210, such as polymer nanoparticles, lipid nanoparticles, and inorganic nanoparticles, are attached to the surface of the microneedle part 130. The powder-attached formulation may be configured such that the particles of the active pharmaceutical ingredient 210 are released by body fluids when the microneedle part 130 is inserted into the body.

The coated formulation may be formed in a structure in which a coating layer is formed on the surface of the microneedle part 130. This coating layer may include a polymer matrix containing the active pharmaceutical ingredient 210, sugars, protein stabilizers, and the like. The coated formulation may be designed such that the active pharmaceutical ingredient 210 is eluted when the microneedle part 130 is inserted into the body.

In the microneedle patch 100 according to an embodiment of the present disclosure, the polymer fiber layer 140 may be formed to cover one surface of the microneedle part 130 that is formed in various formulations such as a solid formulation, a dissolvable formulation, a powder-attached formulation, and a coated formulation. This polymer fiber layer 140 may stably protect the active pharmaceutical ingredient 210 included in the microneedle part 130 by suppressing direct contact of external moisture or contaminants with the surface of the microneedle part 130.

In the microneedle patch 100 according to an embodiment of the present disclosure, the polymer fiber layer 140 may be provided in a form to cover and protect the needles 120 of the microneedle part 130 by being electrospun onto one surface of the base 110 of the microneedle part 130. For example, the polymer fiber layer 140 may be formed in a structure that covers the needles 120 on one surface of the base 110. Here, the edge of the polymer fiber layer 140 may be connected to the edge of one surface of the base 110 in a state of contact. In addition, the central part of the polymer fiber layer 140 may be supported in a state of being placed and stacked on the tips of the needles 120. Accordingly, the central part of the polymer fiber layer 140 may be stacked according to the height of the tips of the needles 120 and formed into various shapes such as a flat plate, a dome, or a hemisphere.

More specifically, the polymer fiber layer 140 of the microneedle patch 100 according to an embodiment of the present disclosure may be formed in a tent-like shape that covers and protects the plural needles 120 and the base 110 by being stacked on the plural needles 120 and the base 110 by an electrospinning method, rather than being directly coated on them.

Meanwhile, in the microneedle patch 100 according to an embodiment of the present disclosure, the polymer fiber layer 140 may include a biocompatible polymer. For example, the polymer fiber layer 140 may be formed in a stacked structure of a porous mesh or film including pores. In particular, since the microneedle patch 100 according to an embodiment of the present disclosure is directly inserted into the skin, it is desirable to use a biocompatible polymer to minimize inflammatory and toxic reactions in the body when using the microneedle patch 100. As such, some materials of the biocompatible polymer also provide biodegradability and, accordingly, are naturally degraded and removed from the body after a certain period of time, thereby ensuring safety and reliability when applied to the human body.

In addition, the polymer fiber layer 140 may further include a lipophilic polymer or a hydrophilic polymer. Preferably, the polymer fiber layer 140 may include a lipophilic polymer. As such, the polymer fiber layer 140 including a lipophilic polymer has low solubility in moisture, which may facilitate structural maintenance even in an environment where external moisture or body fluids are present, and may also suppress the active pharmaceutical ingredient 210 included in the plural needles 120 or the plural needles 120 themselves from being eluted or decomposed by moisture. However, in the microneedle patch 100 according to an embodiment of the present disclosure, the polymer fiber layer 140 may include a hydrophilic polymer instead of a lipophilic polymer, and the mechanical strength, hydrophobicity, moisture permeability, and degradation properties may be appropriately controlled according to the application during the formation process of the polymer fiber layer 140.

In the microneedle patch 100 according to an embodiment of the present disclosure, as the lipophilic polymer usable for the polymer fiber layer 140, polycaprolactone (PCL), polylactic acid (PLA), or the like may be used, and as the hydrophilic polymer, polyvinyl alcohol (PVA), polyethylene oxide (PEO), or the like may be used. However, the present disclosure is not limited thereto, and various polymer materials that exhibit substantially the same or similar effects may be applied.

In addition, in the microneedle patch 100 according to an embodiment of the present disclosure, the polymer fiber layer 140 may have a structure stacked in the form of a porous mesh or film including pores, and may be formed by an electrospinning method. When this electrospinning method is applied, ultrafine fibers of a level of tens to hundreds of nanometers may be manufactured, and by stacking the ultrafine fibers manufactured by the electrospinning method in the form of a mesh or a film, various thicknesses and pore structures may be formed according to design conditions.

For example, in the microneedle patch 100 according to an embodiment of the present disclosure, the thickness of the polymer fiber layer may be 10 to 50 μm, and it may be implemented in the form of a mesh or a film depending upon the electrospinning conditions, fiber arrangement density, stacking thickness, and degree of bonding between fibers. For reference, when the thickness of the polymer fiber layer 140 is less than 10 μm, the blocking effect against external moisture or contaminants may be insufficient, and due to low mechanical stability, the needles 120 are likely to be easily broken or damaged during the insertion process. In addition, when the thickness of the polymer fiber layer 140 exceeds 50 μm, the polymer fiber layer 140 becomes thicker, which increases the insertion resistance of the needles 120, and there is a concern that the skin penetration power of the needles 120 may be reduced.

The mesh form may be a structure in which electrospun fibers are relatively loosely stacked to include pores that are visible to the naked eye. The film form is a structure in which electrospun fibers are densely stacked to form a visually continuous membrane structure, but in reality, it may be a porous sheet in which ultrafine fibers are stacked, maintaining micropores between the fibers. That is, the polymer fiber layer 140 according to an embodiment of the present disclosure may be implemented in a mesh form or a film form according to the characteristics of the external environment. In particular, since the film form exhibits a blocking performance even for nano- to micrometer-sized contaminants such as bacteria or fine particles and thus may more effectively ensure the stability of the active pharmaceutical ingredient 210, a mesh structure or a film structure may be selectively applied according to the size and nature of the external contaminants to be blocked.

The microneedle patch 100 according to an embodiment of the present disclosure may sufficiently ensure the blocking property against external moisture, bacteria, and contaminants by implementing the polymer fiber layer 140 in a mesh or film form, and may also ensure skin permeability by designing the fiber bonds to be easily broken by local stress when the tips of the microneedle part 130 are inserted. Therefore, when the mesh-type polymer fiber layer 140 is formed, a plurality of tips of the microneedle part 130 in the microneedle patch 100 may pass through the polymer fiber layer 140 via the pores and be inserted into the skin. In addition, when the film-type polymer fiber layer 140 is formed, the polymer fiber layer 140 may be locally broken by high local stress generated during skin insertion of the microneedle part 130, so that the insertability of the microneedle patch 100 may be maintained.

In addition, in the microneedle patch 100 according to an embodiment of the present disclosure, the polymer fiber layer 140 formed by electrospinning provides a high specific surface area and porosity, and may increase the hydrophobicity of the fiber layer due to inter-fiber pores and surface roughness. More specifically, the polymer fiber layer 140 formed by electrospinning has a high specific surface area and porosity, and its hydrophobicity may be improved by a plurality of pores formed between fibers and a fine uneven structure on the fiber surface. Therefore, the microstructure formed by electrospinning suppresses the wide spreading of external moisture on the surface of the polymer fiber layer 140 and restricts penetration into the fiber layer by keeping water droplets in a nearly spherical state, thereby stably protecting the active pharmaceutical ingredient 210 included in the microneedle part 130 due to its own improved water-repellent properties.

In addition, in the microneedle patch 100 according to an embodiment of the present disclosure, the polymer fiber layer 140 may suppress contaminants from reaching the surface of the microneedle part 130 by the size of a plurality of pores formed between the fibers and the electrostatic interaction between the fibers and the contaminants. More specifically, in the microneedle patch 100 according to an embodiment of the present disclosure, the pore size of the polymer fiber layer may be about 0.5 to 4 μm.

When the pore size of the polymer fiber layer 140 is less than 0.5 μm, the polymer fiber layer 140 becomes dense overall, increasing the fracture resistance of the polymer fiber layer 140 when the needles 120 are inserted, which may thereby degrade the insertion characteristics. When the pore size of the polymer fiber layer 140 exceeds 4 μm, there is a possibility that external contaminants may pass through the pores and reach the surface of the microneedle part 130. As such, by controlling the size of the plural pores formed between the fibers to be smaller than or similar to the average size of external contaminants in the microneedle patch 100 according to an embodiment of the present disclosure, it is possible to prevent the contaminants from directly reaching the surface of the microneedle part 130 due to a physical filtration effect.

Furthermore, since the surface of the fibers of the polymer fiber layer 140 formed during the electrospinning process may retain an electrostatic charge, contaminants in the form of fine particles introduced from the outside are adsorbed or repelled by electrostatic attraction or repulsion on the fiber surface, which may provide an additional blocking effect. Therefore, the polymer fiber layer 140 may perform a complex blocking function based on the pore size and surface charge characteristics, and may stably protect the active pharmaceutical ingredient 210 included in the microneedle part 130 from external contaminants.

In addition, in the microneedle patch 100 according to an embodiment of the present disclosure, the polymer fiber layer 140 may further include a hygroscopic material. Through this, the polymer fiber layer 140 may suppress the denaturation of the active pharmaceutical ingredient 210 and the denaturation of the microneedle part 130 due to moisture absorption during long-term storage. For example, a moisture adsorbent such as silica gel may be used as the hygroscopic material of the microneedle patch 100 according to an embodiment of the present disclosure, but the configuration of the present disclosure is not limited thereto.

When a hygroscopic material is introduced into the polymer fiber layer 140 as described above, trace amounts of moisture introduced from the outside are immediately adsorbed and captured inside the polymer fiber layer 140, thereby reducing the relative humidity and water activity in the surrounding environment of the microneedle part 130. Therefore, during long-term storage, the hydrolysis, oxidation, and aggregation of the active pharmaceutical ingredient 210, the dulling, deformation, and fusion of the tips of the microneedle part 130, and the elution and recrystallization of the coating layer due to moisture absorption may be suppressed.

In addition, the polymer fiber layer 140 formed by electrospinning may have the characteristic that a moisture penetration path is blocked by being imparted with hydrophobicity due to the pores between the fibers and the fine unevenness of the fiber surface. Therefore, when the polymer fiber layer 140 of this embodiment further includes a hygroscopic material, it may more effectively suppress external moisture from directly reaching the surface of the microneedle part 130 by combining a blocking function based on low wettability on the surface and an absorption function based on moisture capture inside. At this time, in the microneedle patch 100 according to an embodiment of the present disclosure, the effect on the long-term storage stability of the microneedle patch 100 by the hygroscopic material is a general phenomenon based on the physical properties of the hygroscopic material, but it is not limited to the embodiment of the present disclosure and may be understood from common technical knowledge.

Hereinafter, a method of manufacturing a microneedle patch according to another embodiment of the present disclosure will be described in detail.

The method of manufacturing a microneedle patch according to another embodiment of the present disclosure may include: a step of forming the microneedle part 130 including the base 110 and the plural needles 120 for forming tips protruding in a sharp shape from one surface of the base 110; and a step of forming the polymer fiber layer 140 that is formed to cover the microneedle part 130 by electrospinning a lipophilic and biocompatible polymer solution onto the microneedle part 130 to perform the function of protecting the active pharmaceutical ingredient 210 included in the needles 120 from moisture and contaminants.

In the step of forming the microneedle part, the microneedle part may be formed using various technologies known in the art, and may be formed of at least one of a solid formulation, a dissolvable formulation, a powder-attached formulation, and a coated formulation. For example, the microneedle part may be manufactured through general processes such as micromold casting, photopolymerization, additive manufacturing (3D printing), or injection and drying of a polymer solution containing pharmaceutical ingredients, and the configuration of the present disclosure is not limited to a specific manufacturing method.

In the step of forming a polymer fiber layer, it is desirable to use a polymer solution that is a lipophilic and biocompatible polymer, and specific examples such as polycaprolactone (PCL) and polylactic acid (PLA) may be used. A lipophilic polymer has low solubility in moisture, which may facilitate structural maintenance of the polymer fiber layer even in an environment where external moisture or body fluids are present. As a result, it may be possible to suppress pharmaceutical ingredients contained in the microneedle part or the microneedle part itself from being eluted or decomposed by moisture.

However, in the method for manufacturing a microneedle patch, the polymer fiber layer may be formed using not only a lipophilic polymer but also a hydrophilic polymer, and in the step of forming the polymer fiber layer, mechanical strength, hydrophobicity, moisture permeability, and degradation properties may be adjusted according to the application. For example, as the hydrophilic polymer usable in the step of forming the polymer fiber layer, polyvinyl alcohol (PVA), polyethylene oxide (PEO), or the like may be used, but the configuration of the present disclosure is not limited thereto, and various polymer materials that may exhibit substantially the same or similar effects are applicable.

Meanwhile, in the step of forming the polymer fiber layer, the polymer solution may include a cosolvent to ensure the shape stability and fiber uniformity of the polymer fiber layer. More specifically, the polymer solution for forming a polymer fiber layer may be prepared by mixing two or more types of solvents having different degrees of polarity. It dissolves well in weakly polar solvents (e.g., acetone, THF, etc.), but during electrospinning, the solvent evaporates quickly, which may cause fibers to break or beads to form. In addition, when only a highly polar solvent is used, the evaporation of the solution is slow, so the fibers may be deposited in a wet state, forming a tough film.

Therefore, in the step of forming the polymer fiber layer of the method of manufacturing a microneedle patch according to another embodiment of the present disclosure, a cosolvent system in which a first solvent with relatively low polarity and a fast evaporation rate and a second solvent with relatively high polarity and a slow evaporation rate are mixed is applied, so that the solubility of the polymer and the viscosity of the solution may be stably maintained. For example, by combining solvents with different evaporation rates, it is possible to ensure the continuity of the solution jet while fibers are formed during the spinning process and to control the formation of fibers with a uniform diameter distribution.

In another embodiment of the present disclosure, the cosolvent may include acetone and acetic acid. Preferably, the composition ratio of acetone and acetic acid may be 7:3 to 9:1. That is, by using a polymer solution including a cosolvent, the polymer fiber layer may be formed in a structure having a uniform fiber diameter and a smooth surface, and may stably maintain a barrier function that blocks external moisture or contaminants. In addition, by using a polymer solution including a cosolvent, a network structure between fibers is homogeneously formed, so that local fracture characteristics are kept constant when using the microneedle patch, thereby securing both the function of the protective layer and the insertion characteristics of a microneedle part at the same time.

In the step of forming the polymer fiber layer of the method of manufacturing a microneedle patch according to another embodiment of the present disclosure, the drying rate and deposition pattern of the fibers during the electrospinning process may be controlled by adjusting the composition ratio of the cosolvent, and thereby, the shape stability of the fiber layer may be improved to form a uniform the polymer fiber layer 140 on one surface of a microneedle part.

In addition, in the method of manufacturing a microneedle patch according to another embodiment of the present disclosure, the step of forming the polymer fiber layer may include electrospinning the polymer solution at an electrospinning speed of 8 to 10 mL/h. When spinning at a speed of less than 8 mL/h, it may be difficult to form uniform fibers due to an insufficient amount of the spun polymer solution. When spinning at a speed exceeding 10 mL/h, the amount of the spun polymer solution is excessive, so a thick and non-uniform fiber layer may be formed, and the solvent may not volatilize sufficiently, which may cause filming of the polymer solution or the formation of droplets. The polymer fiber layer formed at an electrospinning speed of 8 to 10 mL/h may secure a uniform pore structure, thereby maximizing the effect of blocking moisture and contaminants.

In the method of manufacturing a microneedle patch according to another embodiment of the present disclosure, the polymer fiber layer 140 may be formed in a shape that covers and protects one surface of the microneedle part 130 through an electrospinning process. For example, the edge of the polymer fiber layer 140 may be connected by contact to the edge of one surface of the base 110 of the microneedle part 130, and the central part of the polymer fiber layer 140 may be supported in a stacked structure where it is placed on the tips of the needles 120 of the microneedle part 130.

Since a general electrospinning process involves random deposition of fibers on a flat substrate, a problem where the fibers are locally concentrated on the tips or specific areas of the microneedle part, or are not sufficiently formed on the base may arise, when applied to a microneedle part having a three-dimensional structure. This problem causes limitations in securing the continuity and uniformity of the protective layer, which may impair the external blocking function and mechanical properties upon insertion. However, in the step of forming the polymer fiber layer of the method of manufacturing a microneedle patch according to another embodiment of the present disclosure, it is possible to induce the fibers to be uniformly deposited on one surface of the microneedle part and to control the polymer fiber layer to form a tent-like structure that covers and wraps the plural needles, by controlling the arrangement of the collector equipped with the microneedle part and the electric field conditions.

The present disclosure has an advantage in that a uniform and continuous fiber layer can be formed over one surface of the three-dimensional structure of the microneedle part by controlling the electrospinning conditions and collector arrangement. In addition, since the polymer fiber layer formed by the electrospinning process has the form of a porous mesh or film including a plurality of pores, the microneedle part can easily penetrate the fiber layer by local pressure during skin insertion, and the performance of blocking contaminants by the polymer fiber layer and the insertion characteristics of the microneedle part can be secured at the same time.

Hereinafter, the present disclosure will be described in more detail through examples. These examples are intended to explain the present disclosure more specifically, and the scope of the present disclosure is not limited by these examples.

### [Preparation Example 1] Preparation of Polycaprolactone (PCL)

A polycaprolactone (PCL) solution was prepared by dissolving PCL (80 kDa) at a concentration of 10 w/v % using a cosolvent in which acetone and acetic acid were mixed at a ratio of 8:2. The PCL solution was dissolved by stirring with a magnetic stirrer for 2 hours at 40° C.

The prepared PCL solution was injected into a 10 ml syringe, which was then equipped with a 21G metal nozzle and connected to an electrospinning apparatus (nanoNC, ESR200R2). Electrospinning was performed under the conditions of a voltage of 18 to 20 kV, a solution injection speed of 8 to 10 mL/h, and a distance of 10 cm between the nozzle and the collector.

The electrospinning was performed at 25° C. and 40% relative humidity, and was carried out using aluminum foil as a collector. The PCL fibers collected on the aluminum foil were naturally dried at room temperature (25° C.) to obtain a final fiber form.

### [Experimental Example 1] Evaluation of Microstructure and Porosity of Polycaprolactone (PCL) Fibers

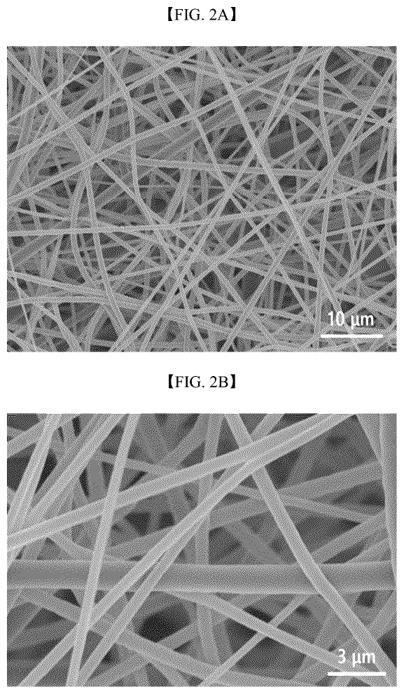

The PCL fibers prepared by electrospinning were sputter-coated with platinum (Pt) before analysis, and their microstructure was observed at various magnifications using a scanning electron microscope (SEM, SU8600, Hitachi). The obtained SEM images were binarized using ImageJ software, and then the size and porosity of the pores were calculated through area analysis of the pore regions, with the results shown in FIGS. 2A, 2B and FIG. 3.

FIGS. 2A and 2B illustrate the images of PCL fibers electrospun according to the method of the preparation example, observed with a scanning electron microscope (SEM). FIG. 2A shows the overall structure of the electrospun fiber layer taken at a magnification with a 10 μm scale bar, and FIG. 2B shows the detailed structure of the fibers taken at a magnification with a 3 μm scale bar.

As shown in FIGS. 2A and 2B, the PCL fibers electrospun according to the method of the preparation example have a network structure formed by randomly entangled fine fibers, and it can be confirmed that the pores between the fibers are uniformly distributed.

FIG. 3 illustrates the results of a quantitative analysis of the pore size distribution of the electrospun PCL fiber layer observed in FIGS. 2A and 2B. The analysis was performed by measuring the individual pore size between fibers in the SEM image using ImageJ software, followed by statistical processing, and it shows the frequency (%) according to the pore size.

As shown in FIG. 3, about 40% of the total pores are distributed in the 0.5 μm size range, the 1.0 μm range accounts for about 25%, and the 1.5 μm range accounts for about 20%, confirming that most of the pores are concentrated at 1.5 μm or less. The average pore size was calculated to be about 1.13 μm, and the total porosity was confirmed to be about 40.5%.

The result of FIG. 3 is interpreted as showing that the PCL fiber layer has structural characteristics that can simultaneously perform the function of a physical barrier capable of suppressing the penetration of external particulate contaminants and moisture, while maintaining high permeability to allow the tips of the microneedle part to pass through the pores and be inserted into the skin.

### [Experimental Example 2] Evaluation of Surface Wettability (Contact Angle) of PCL Fibers

A solution with the same composition as the one used to prepare the PCL fibers was dispensed in an amount of 1 mL into an aluminum dish, and then naturally dried at room temperature for 12 hours or more to form a solid PCL film.

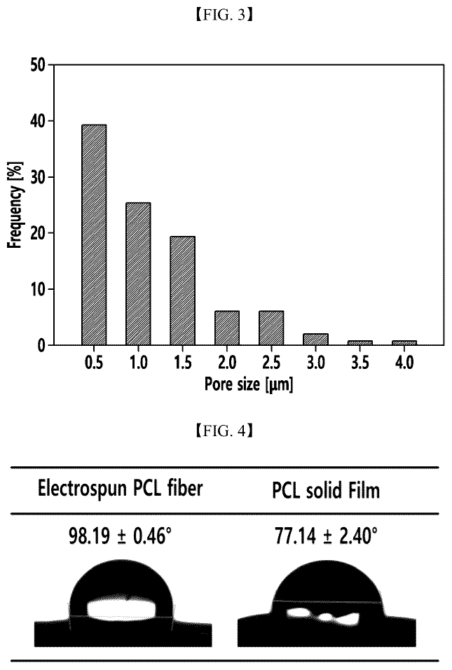

To compare the surface properties of the PCL fibers electrospun according to the preparation example and the solid film, a contact angle analyzer (Phoenix-10, SEO Co., Ltd.) was used, and the static contact angle was measured by dropping 1 μL of distilled water on the surface of each sample, with the results shown in FIG. 4.

FIG. 4 illustrates the results comparing the surface contact angle of PCL fibers electrospun according to the preparation example with that of a solid film prepared from a PCL solution of the same composition.

As shown in FIG. 4, the electrospun PCL fiber on the left exhibits a contact angle of about) 98.19° (±0.46°, and since the water droplet on the surface does not spread and maintains a spherical shape, it is interpreted as having high hydrophobicity.

The electrospun PCL fiber is interpreted as having high hydrophobicity because the penetration of water molecules is suppressed and the surface tension is maintained due to the porous fiber structure formed by the electrospinning process and surface roughness.

The PCL solid film on the right is observed to have a contact angle of about) 77.14° (±2.40°. Since the PCL solid film has a flat structure without pores, it is analyzed to have lower hydrophobicity than the electrospun PCL fiber because water makes wide contact with the surface, promoting the wetting phenomenon.

Therefore, it can be confirmed that the electrospun PCL fiber layer, despite being of the same material, has higher hydrophobicity compared to the solid film, and thus can effectively protect the active ingredients of the microneedle part from external moisture, and is applicable to microneedle parts, made of various formulations, due to the flexibility resulting from its porous structure.

### [Experimental Example 3] Evaluation of Mechanical Properties of PCL Fibers

PCL fiber samples of various thicknesses were prepared by electrospinning a 10 w/v % PCL solution while controlling the total spinning amount. The prepared fibers were cut into a size of 2 cm×2 cm, and then their mechanical properties were measured using a stress-strain tester (ZwickiLine, ZwickRoell). The measurement was performed at a tensile speed of 2 mm/min while applying a load to the sample at an angle of 90°, and the Young's modulus was calculated based on an initial linear section, and an elongation at break was also evaluated.

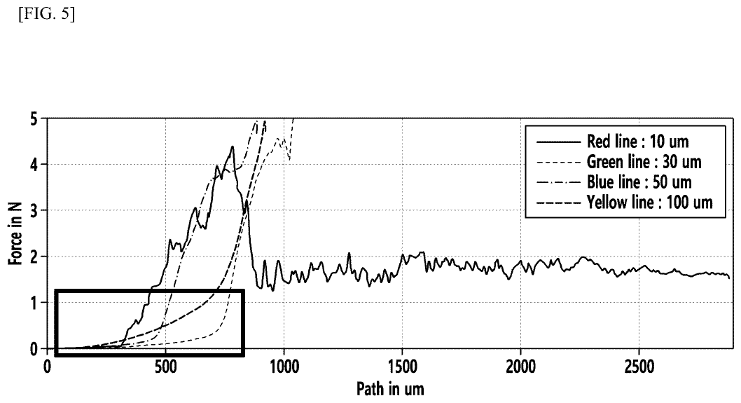

FIG. 5 is a set of stress-strain curves illustrating the mechanical properties of PCL fibers of various thicknesses prepared by controlling the spinning amount of a polycaprolactone (PCL) solution, and Table 1 below shows the Young's modulus and elongation at break dependent upon the PCL fiber thickness, calculated according to FIG. 5.

TABLE 1

| Fiber thickness (μm) | Young's modulus (Pa) | Elongation (%) |
|---|---|---|
| 10 | 10.06 ± 6.05 | 5564 ± 464 |
| 30 | 32.55 ± 3.35 | 2525 ± 129 |
| 50 | 39.07 ± 3.63 | 1861 ± 54 |
| 100 | 255.58 ± 44.43 | 748 ± 78 |

The graph of FIG. 5 shows a comparison of the behavior when the fiber thickness is 10 μm (red line), 30 μm (green line), 50 μm (blue line), and 100 μm (yellow line), respectively.

As shown in FIG. 5, as the fiber thickness increases, the initial slope of the stress-strain curve (Young's modulus) significantly increases, whereas the elongation at break tends to decrease sharply.

In particular, in the case of the 10 μm thick fiber (red line), the initial slope is gentle, indicating a low Young's modulus, and as the stress gradually increases, it is continuously measured without fracture even up to a deformation path of 2500 μm or more, which is confirmed to have high ductility and a wide deformation range.

Conversely, the 100 μm-thick fiber (yellow line) fractures immediately after the stress rapidly increases in a short deformation path, which means brittle behavior with both a high Young's modulus and low elongation.

According to the results of FIG. 5, it can be confirmed that the fiber thickness directly affects the mechanical properties, with thin fibers providing excellent ductility and flexibility, and thick fibers providing high stiffness and durability. Therefore, it can be seen that by controlling the thickness of the fibers according to the application purpose, a balance between mechanical flexibility and structural stability can be secured.

### [Preparation Example 2] Preparation of Microneedle Part Protected with PCL Mesh

A PCL solution was prepared by dissolving polycaprolactone (PCL, 80 kDa) at a concentration of 10 w/v % using a cosolvent in which acetone and acetic acid were mixed at a ratio of 8:2. The PCL solution was dissolved by stirring with a magnetic stirrer for 2 hours at 40° C.

The prepared PCL solution was injected into a 10 mL syringe, which was then equipped with a 21G metal nozzle and connected to an electrospinning apparatus (nanoNC, ESR200R2). Electrospinning was performed under the conditions of a voltage of 18 to 20 kV, a solution injection speed of 8 to 10 mL/h, and a distance of 10 cm between the nozzle and the collector, and was carried out at 25° C. and 40% relative humidity.

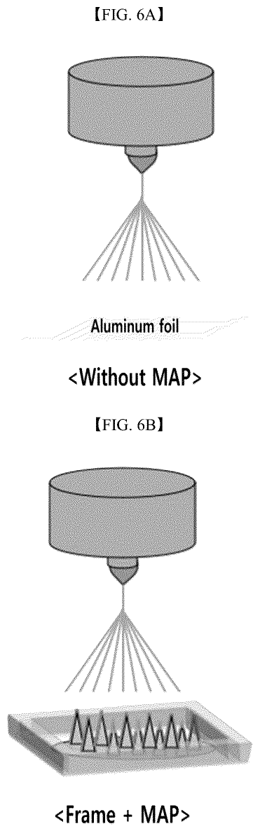

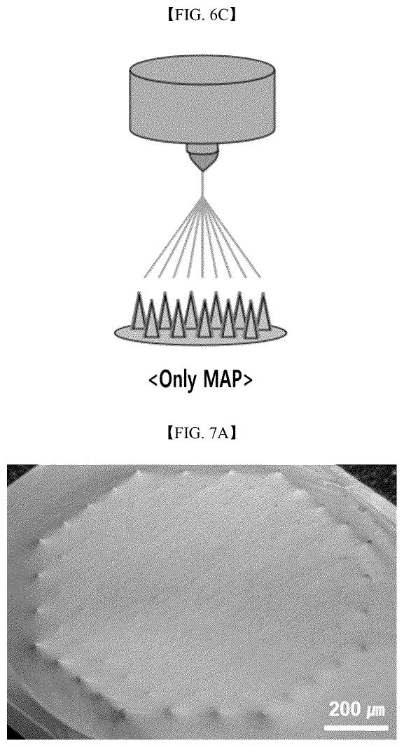

The electrospinning process was performed as shown in the schematic view of the electrospinning process depending upon the presence or absence of a conductive frame and a microneedle part, as shown in FIGS. 6A to 6C, and the specific conditions are as described in Table 2.

TABLE 2

| | Preparation | Preparation | Preparation |
|---|---|---|---|
| Classification | Example 2-1 | Example 2-2 | Example 2-3 |
| Presence/absence of | ◯ | X | ◯ |
| conductive frame | | | |
| Presence/absence of | X | ◯ | ◯ |
| microneedle part | | | |
| Sample name | Without MAP | Only MAP | Frame + MAP |

A microneedle part in the form of a monument with a height of 800 μm, an interval of 470 μm, and a width of 350 μm was used, with a total of 97 needles arranged on a circular base with a diameter of 1 cm, and spinning was repeated for about 2 minutes while periodically observing whether the PCL fibers covered the microneedle part during electrospinning. After the electrospinning was completed, the sample was naturally dried at room temperature.

### [Experimental Example 4] Optimization of Thickness of PCL Mesh for Protecting Microneedle Part

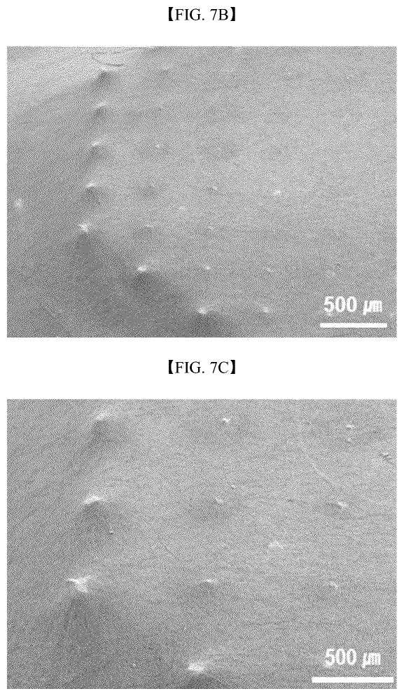

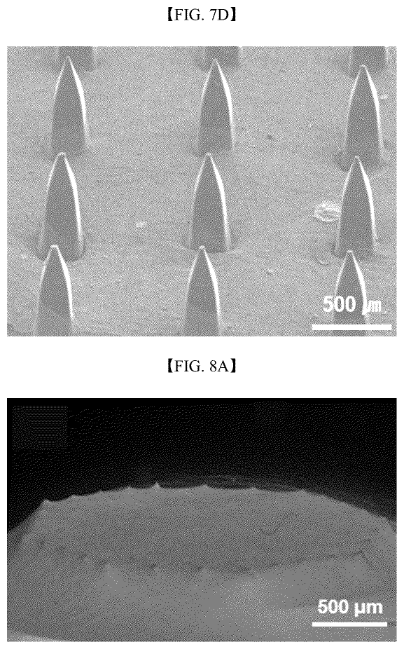

FIGS. 7A to 7D are a set of images illustrating the structure of an electrospun fiber layer formed on the top of the microneedle part according to an embodiment of the present disclosure and the insertability of the microneedle part.

FIGS. 7A to 7C are images observing the electrospun PCL fiber layer formed on top of a microneedle part with an optical microscope (FIG. 7A) and a scanning electron microscope (SEM, FIGS. 7B and 7C), respectively, from which it can be confirmed that the electrospun PCL fiber layer formed a protective layer that uniformly covers the entire top of the microneedle part.

In particular, FIG. 7C is an image observing the electrospun PCL fiber layer formed on the top of the microneedle part at a wider magnification, and it is confirmed that the tips of the microneedle part are locally protruded even when the electrospun fiber layer is formed.

The image of FIG. 7C is interpreted as showing that the electrospun fiber layer has inter-fiber pores and mechanical flexibility, and it is interpreted that the electrospun fiber layer will be locally broken during the insertion process of the microneedle part or the tips of the microneedle part will pass through the pores and be inserted into the skin.

FIG. 7D is an SEM image of a microneedle part that has passed through the PCL electrospun fiber layer, and since the tips of the microneedle part clearly protrude even with the protective layer formed, it is interpreted that the tips of the microneedle part can sufficiently penetrate the skin tissue even during skin insertion with the protective layer formed.

Therefore, FIGS. 7A to 7D confirms that the electrospun fiber layer of the present disclosure effectively protects the upper part of the microneedle part while maintaining insertability, and it can be seen that both the protective function and the insertion function were secured.

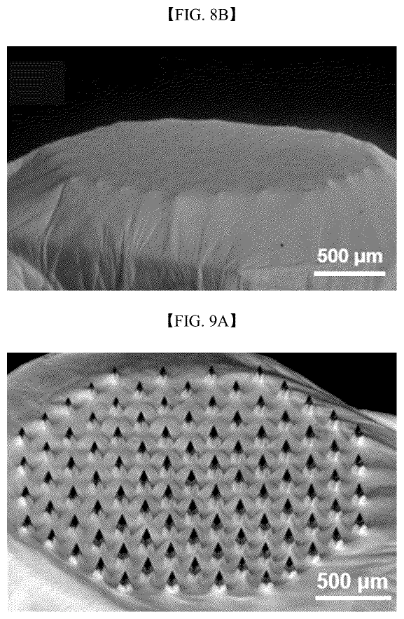

FIGS. 8A and 8B illustrates a set of optical microscope images of a polycaprolactone (PCL) mesh formed on the top of a microneedle part, dependent upon the presence or absence of a conductive frame, and Table 3 below shows the results of quantitatively measuring the thickness of the PCL mesh dependent upon the presence or absence of the conductive frame.

TABLE 3

| Classification | PCL mesh thickness (μm) |
|---|---|
| Only MAP (Example 1) | 18.42 ± 4.66 |
| Frame + MAP (Example 2) | 30.42 ± 5.22 |
| Without MAP | 197.58 ± 23.38 |

In the case of FIG. 8A using only a non-conductive microneedle part without a conductive frame (only MAP), the fibers were formed in a non-uniformly dispersed manner even under the same conditions, and it is confirmed that they do not adhere evenly onto a microneedle part, and the thickness of the formed mesh is also thin.

The thickness of the PCL mesh formed when using only the non-conductive microneedle part of FIG. 8A was measured to be about 18.42±4.66 μm on average, and in the edge area of the microneedle part, phenomena where fibers were formed non-uniformly or some of the microneedle part was exposed without protection were observed.

On the other hand, in the case of FIG. 8B in which a microneedle part was placed within a conductive frame (Frame+MAP), fibers were uniformly deposited as the electric field was stably formed due to the presence of the conductive frame, and a stably adhered PCL mesh was formed over the entire upper part of the microneedle part.

The thickness of the PCL mesh formed when the microneedle part was placed within the conductive frame was measured to be about 30.42±5.22 μm, from which it can be seen that the mesh thickness was significantly increased by the introduction of the conductive frame.

In addition, when only a conductive frame and a conductive substrate (aluminum foil) were used without a microneedle part (without MAP), fibers were intensively deposited in a narrow area, and the PCL mesh was formed to be significantly thicker, with an average thickness of about 197.58±23.38 μm.

The thick formation of the PCL mesh on the conductive frame is interpreted as a result of the dispersion of the electric field being suppressed due to the absence of a microneedle part, leading to the intensive stacking of fibers.

Therefore, from the results of FIGS. 8A and 8B, it can be confirmed that when a conductive frame is introduced in the electrospinning process, the instability of the electric field generated on a non-conductive substrate is effectively compensated for, and as a result, a protective layer with uniform fiber deposition and secured reproducibility is formed on the top of the microneedle part.

### [Example 2] Manufacture of Microneedle Part (E-MAP) Protected with PCL Mesh

A polycaprolactone (PCL) solution was prepared by dissolving PCL (80 kDa) at a concentration of 10 w/v % using a cosolvent in which acetone and acetic acid were mixed at a ratio of 8:2. The PCL solution was dissolved by stirring with a magnetic stirrer for 2 hours at 40° C.

The prepared PCL solution was injected into a 10 ml syringe, which was then equipped with a 21G metal nozzle and connected to an electrospinning apparatus (nanoNC, ESR200R2). Electrospinning was performed by placing a microneedle part within a conductive frame, under the conditions of a voltage of 18 to 20 kV, a solution injection speed of 8 to 10 mL/h, and a distance of 10 cm between the nozzle and the collector. The electrospinning was performed at 25° C. and 40% relative humidity.

By controlling the spinning time, microneedle parts (E-MAP) were fabricated with PCL meshes with 10, 30, 50, and 100 μm thickness formed as a protective layer.

### [Experimental Example 5] Confirmation of Exposed Height of Tips of Microneedle Part Dependent Upon PCL Mesh Thickness

In the present disclosure, an experiment was conducted to verify the effective area for drug delivery by evaluating the effective height (penetration height) at which a microneedle part can be inserted into the actual skin after penetrating a PCL mesh formed as a protective layer.

To simulate mechanical properties similar to skin, a PDMS (Sylgard 184) model was fabricated. The PDMS model was completed by mixing a PDMS base and a curing agent at a ratio of 10:1 (w/w), dispensing the mixture onto a slide glass to a thickness of 1 mm, and then curing at 70° C. for 1 hour.

A microneedle part array (E-MAP) with a PCL mesh of various thicknesses was inserted into the fabricated PDMS model with a force of 5 kg in a vertical direction and then removed, and the tip height of the microneedle part exposed after passing through the PCL mesh was measured using an optical microscope. The measurement was performed by selecting the tips of one microneedle part from the central area and two from the edge areas within the same sample, and the average value was calculated through repeated measurements at each position.

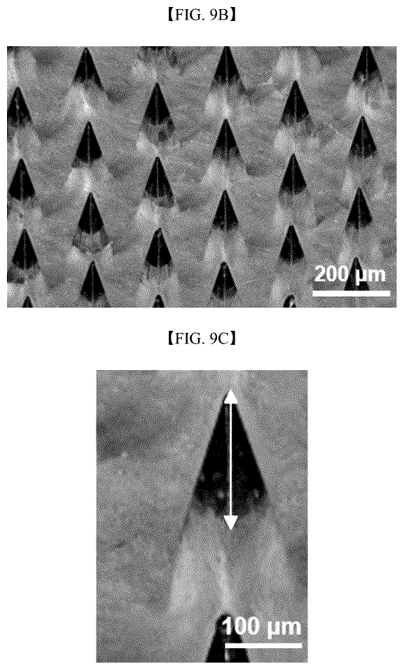

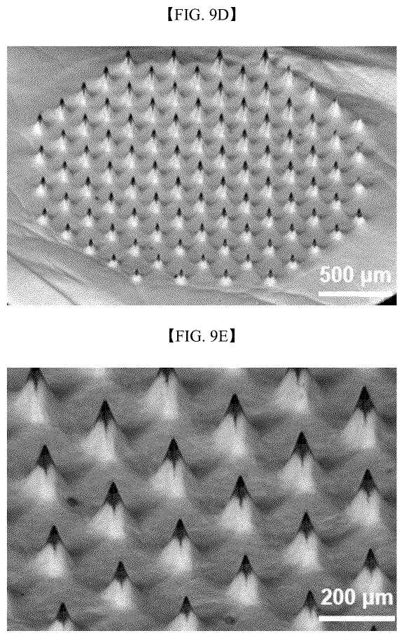

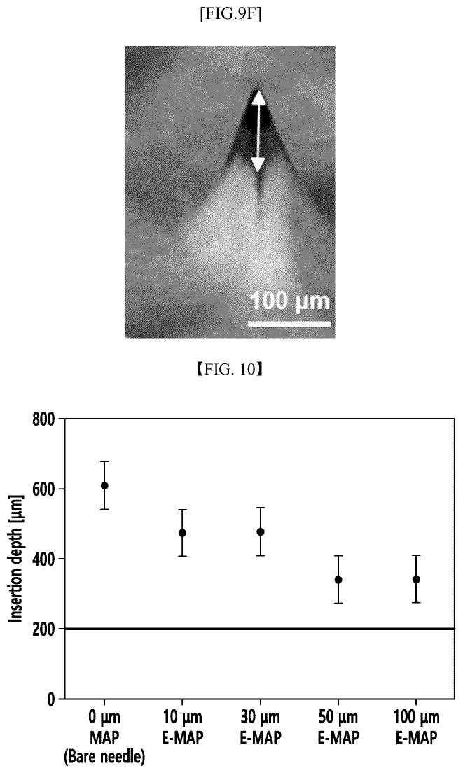

FIGS. 9A to 9F are a set of optical microscope images illustrating a difference in the penetration heights of tips of a microneedle part, exposed when it penetrates a skin simulant (polydimethylsiloxane, PDMS), dependent upon a change in the thickness of a PCL mesh formed through an electrospinning process. FIGS. 9A to 9C show the case where a PCL mesh with a thickness of about 10 μm is applied, and FIGS. 9D to 9F show the case where a PCL mesh with a thickness of about 100 μm or more is applied, respectively.

As shown in FIGS. 9A to 9F, when the thickness of the electrospun PCL mesh is thin (about 10 μm), the tips of a microneedle part are exposed by about 380 μm, indicating a deep insertion depth, whereas as the thickness increases, the exposed height of the microneedle part decreases, and it is confirmed that only about 300 μm of the tips are exposed for a 30 μm thickness, and about 150 μm for a 100 μm thickness.

The result of FIGS. 9A to 9F are interpreted to mean that the insertion depth of the microneedle part is limited by the tension generated when the PCL mesh, formed as a protective layer, creates an arch-shape by adhering to the needle surface but not completely to the base portion during the insertion process of the microneedle part.

In addition, as the thickness of the mesh increases, the stiffness of the electrospun fiber layer increases, and the contact area with the needles increases, leading to a rise in the initial penetration resistance and frictional force. Accordingly, it is analyzed that the penetration depth gradually decreases even under the same load.

Therefore, it can be understood that the thickness of the protective layer formed by electrospinning directly affects the insertion depth of the microneedle part, and it is desirable to design the thickness of the protective layer and the coating height in correlation with each other to maximize the drug delivery efficiency.

### [Experimental Example 6] Evaluation of Microneedle Part Insertion Depth Dependent Upon PCL Mesh Thickness

To evaluate the insertion depth of E-MAP dependent upon a PCL mesh thickness (10, 30, 50, 100 μm), Parafilm M® (Bemis, USA) was used as a skin simulant. Parafilm was prepared by stacking 8 layers, so that the total thickness was about 1,008 μm (126 μm×8 layers).

After fixing the E-MAP vertically on top of the Parafilm, it was attached for 10 seconds with a load of about 5 kg and then removed. After removal, the Parafilm layers were separated one by one, the insertion marks of the microneedle part remaining on each layer were observed with an optical microscope, and the insertion depth of the microneedle part was calculated based on the cumulative depth to the lowermost layer where insertion marks were present.

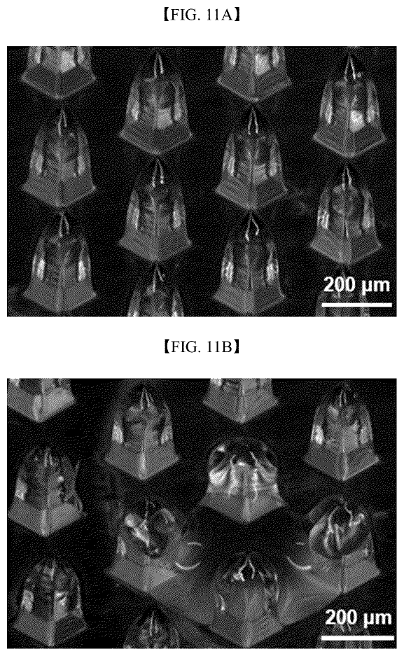

FIG. 10 is a graph illustrating the results of measuring the insertion depths of a microneedle part without a protective layer (bare needles) and a microneedle part with a protective layer (E-MAP) through in-vitro insertion experiments using Parafilm M®.

As shown in FIG. 10, both the bare needles without a protective layer and the E-MAP with a PCL mesh (protective layer) of various thicknesses have an insertion depth of about 270 μm or more, which is a depth exceeding the typical epidermal thickness (about 100-200 μm), confirming a level at which they can successfully penetrate the skin and reach the dermis layer.

In addition, in the case of E-MAP, a trend is observed where the insertion depth of the microneedle part gradually decreases as the PCL mesh thickness increases. For example, when the mesh thickness was 10 μm, the insertion depth was measured to be about 500 μm, whereas with a 100 μm mesh, the insertion depth was confirmed to be limited to around 300 μm.

The result of FIG. 10 is attributed to the structural factor that the PCL mesh does not completely adhere to one surface of the base during the insertion process of the microneedle part, but instead forms a tent-like shape along the tips of the microneedle part, thereby causing mechanical resistance upon insertion.

Therefore, as the mesh gets thicker, the insertion depth is limited, but at the same time, the mesh performs a protective function against moisture and external particles, so it can be understood that a design for optimization between insertion depth and protective function is necessary.

In an embodiment of the present disclosure, the optimal mesh thickness was set to about 30±5 μm, considering the balance between an insertion depth and a protective function.

### [Example 3] Manufacture of Coated Microneedle Part Protected with PCL Mesh (E-C-MAP)

A coating solution was prepared by dissolving 6 w/v % carboxymethyl cellulose (CMC) in distilled water, and then adding either trypan blue or fluorescein isothiocyanate (FITC, 4,000 Da) to a final concentration of 2 w/v %. Trypan blue was used as an indicator for visual confirmation of the coated area, and FITC was used as a reagent for coating confirmation via fluorescence imaging. The remaining volume was supplemented with distilled water to adjust the total composition of the solution.

For surface modification of the microneedle part, an uncoated microneedle part was irradiated with UV/O₃ for 5 minutes using a UV-ozone cleaner. Subsequently, the prepared coating solution was applied to the tips of the microneedle part by a dip-coating method. The dip-coating was performed by repeating the process of immersing the tips 3 times for 1.5 seconds at a speed of 20 mm/s using a well with a depth of 400 μm, followed by drying for 10 minutes, for a total of 6 repetitions. Afterward, the sample was additionally naturally dried at room temperature for 1 hour.

Finally, a polycaprolactone (PCL) mesh with a thickness of about 30±5 μm was formed on the top of the microneedle part using an electrospinning process to protect it.

### [Experimental Example 7] Evaluation of Protective Ability from Moisture

### (1) Observation of Physical Damage to Microneedle Part-Coating Layer

After fixing an E-C-MAP, on which a polycaprolactone (PCL) mesh with an average thickness of 30±5 μm was formed, to a Petri dish with double-sided tape, water was sprayed twice with a sprayer from a horizontal direction at a distance of 20 cm (10 μl per unit area). Next, whether the surface coating of the microneedle part was damaged was observed using an optical microscope and a confocal laser scanning microscope (Nikon Eclipse C1si).

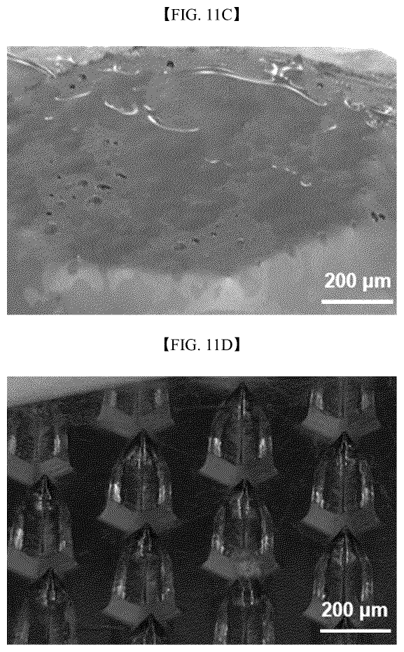

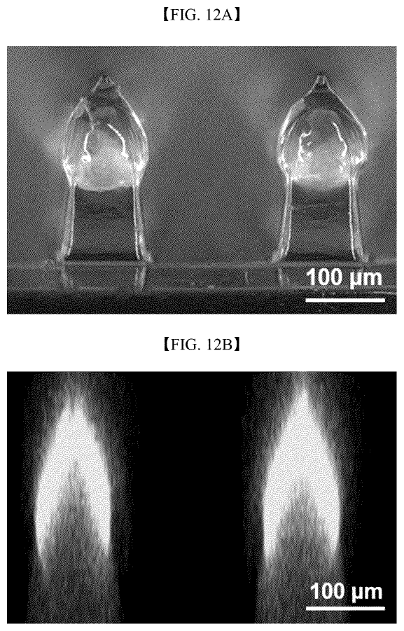

FIGS. 11A to 11D illustrates the results of evaluating the moisture protection ability of a PCL mesh protective layer for a microneedle part coated with trypan blue.

FIG. 11A is an image of a microneedle part coated with trypan blue before water spraying, and FIG. 11B is an image of the case where a water spray treatment was performed on a microneedle part without a protective layer.

As shown in FIG. 11B, when a water spray treatment was performed on a microneedle part without a protective layer, a phenomenon in which a part of the coating layer was detached or damaged was observed. Accordingly, it can be confirmed that the coating layer of the microneedle part and the shape of the microneedle part collapsed due to external moisture.

Meanwhile, FIG. 11C is an image of a PCL mesh protective layer after water-spraying on a microneedle part with a protective layer, and FIG. 11D is an image of a microneedle part with a protective layer under a PCL mesh after water spraying on the microneedle part.

As shown in FIGS. 11C and 11D, in the case of the E-C-MAP to which the PCL mesh protective layer was applied, water droplets were observed to have formed on the PCL mesh surface, and it was confirmed that the microneedle part coated with trypan blue under the PCL mesh protective layer remained undamaged and stable even after the water spray treatment.

Therefore, it is confirmed that the PCL mesh acts as a hydrophobic barrier to effectively protect the coating layer of the inner microneedle part from external moisture, and this is interpreted as simultaneously performing a defensive function against moisture penetration beyond simple structural protection.

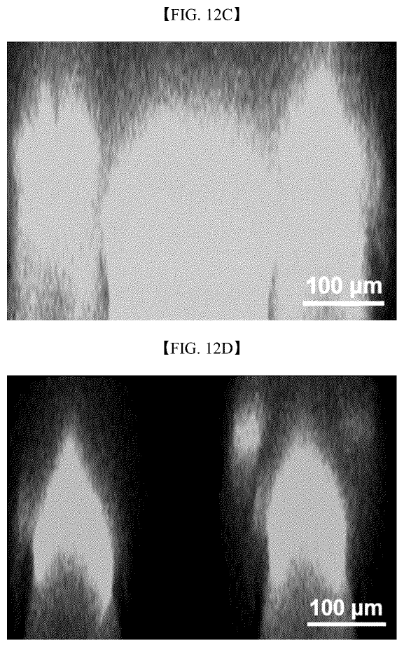

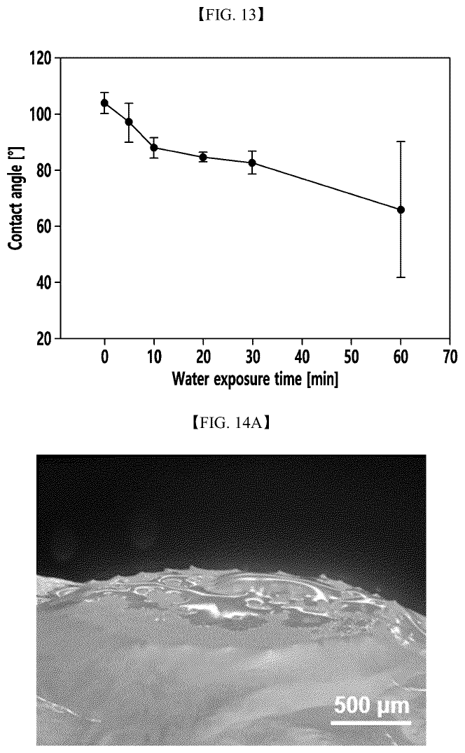

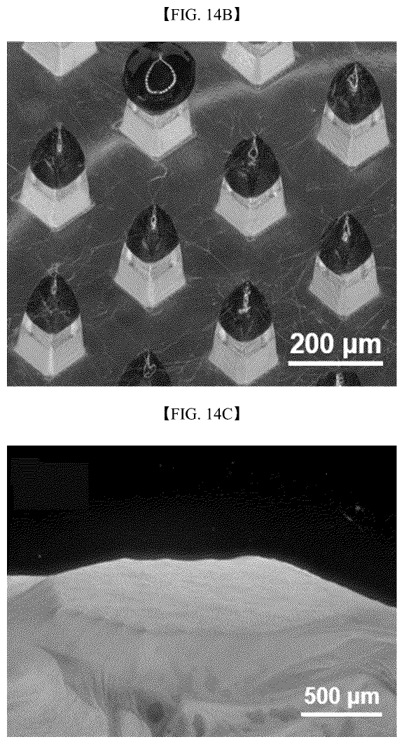

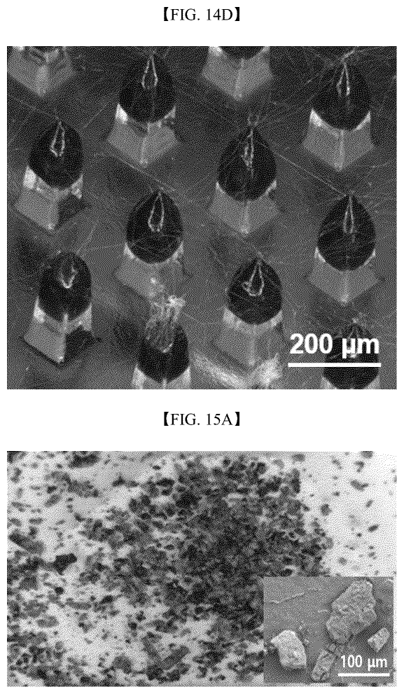

FIGS. 12A to 12D illustrate observation results obtained using confocal laser scanning microscopy to evaluate whether a microneedle part-coating layer is damaged by water spraying.

FIGS. 12A to 12D is an optical microscope image before water spraying, FIG. 12B is a confocal microscope image before water spraying, FIG. 12C is a confocal microscope image after spraying water on a microneedle part without a PCL mesh protective layer (C-MAP), and FIG. 12D is a confocal microscope image after spraying water on a microneedle part with a PCL mesh protective layer applied (E-C-MAP).

As can be confirmed in FIGS. 12A and 12B, the tip area of the microneedle part is uniformly coated with a fluorescent substance (FITC), and the fluorescent signal is clearly maintained, showing that the initial coating layer was stably formed.

As shown in FIG. 12C, in the case of the microneedle part without a PCL mesh protective layer (C-MAP), it is confirmed that the fluorescent signal spreads widely along the surface of the microneedle part after water spraying. The result of FIG. 12C means that the coating layer was dissolved or partially peeled off and damaged by moisture, and it can be confirmed that when a protective layer is absent, the coating layer is easily damaged by external moisture stimulation.

In the case of the microneedle part with a PCL mesh protective layer applied (E-C-MAP) in FIG. 12D, it can be seen that the fluorescent signal is maintained clearly along the shape of the microneedle part overall, although a local defect of the fluorescent signal is observed in some tip areas after water spraying.

From the results in FIGS. 12A to 12D, it is confirmed that the PCL mesh can act as a protective film for the drug coating layer of the microneedle part to suppress the damage and dissolution of the coating layer in an external moisture environment, and it is interpreted that it can ensure the stability of the coating layer and guarantee reproducible drug delivery efficiency in an actual use environment.

### (2) Evaluation of Wettability Change Dependent Upon Moisture Exposure Time

After statically dropping distilled water onto the PCL mesh surface of a coated microneedle part protected with a PCL mesh (E-C-MAP) using a dropper, the change in surface wettability was evaluated by measuring the contact angle according to the exposure time (0, 5, 10, 20, 30, 60 minutes).

FIG. 13 illustrates a change in a contact angle dependent upon the time a PCL mesh is exposed to moisture, and FIGS. 14A to 14D illustrate optical microscope images of a PCL mesh and microneedle part-coating layer dependent upon the time of exposure to moisture.

As shown in FIG. 13, the surface of the PCL mesh in the initial state of moisture exposure showed a contact angle of about 100° or more, exhibiting hydrophobic properties, but the contact angle gradually decreased as the moisture exposure time elapsed. In particular, after 10 minutes, it can be confirmed that the contact angle drops to 90° or less, indicating a clear increase in hydrophilicity.

FIGS. 14A and 14B show optical microscope images of the PCL mesh and the microneedle part-coating layer inside it immediately after moisture exposure (0 min), at which point it is observed that water droplets form on the mesh surface and the coating layer remains stable.

On the other hand, FIGS. 14C and 14D show the results after 10 minutes of moisture exposure, where a pattern of moisture absorption and penetration into the fiber layer appears along with a decrease in the contact angle, and traces of residual moisture are confirmed around the microneedle part-coating layer.

Therefore, through the results of FIGS. 13 and FIGS. 14A to 14D, it can be seen that the surface of the PCL mesh gradually becomes hydrophilic according to the moisture exposure time, and after a certain period, moisture penetrates into the fiber layer, affecting the stability of the microneedle part-coating layer. In addition, it can be confirmed that if it is not removed within 10 minutes, moisture remains or penetrates the surface, causing structural or functional changes.

### [Experimental Example 8] Evaluation of Protective Ability from Contaminants

An E-MAP with an average thickness of 30±5 μm was fixed to a Petri dish using double-sided tape. Particle samples with average particle sizes of 300, 125, 70, and 20 μm, respectively, were first observed for shape and size using SEM.

Next, the corresponding particles were scattered on the surface of the E-MAP fixed in the Petri dish, and then shaken 10 times each in the vertical and horizontal directions to induce particle penetration. After the experiment was completed, the PCL mesh was removed, and the presence of penetrated particles was confirmed through an optical microscope.

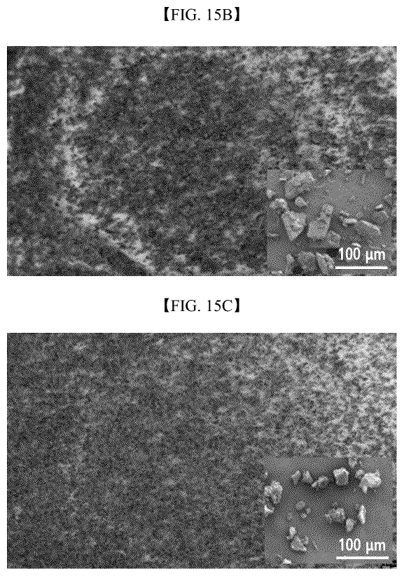

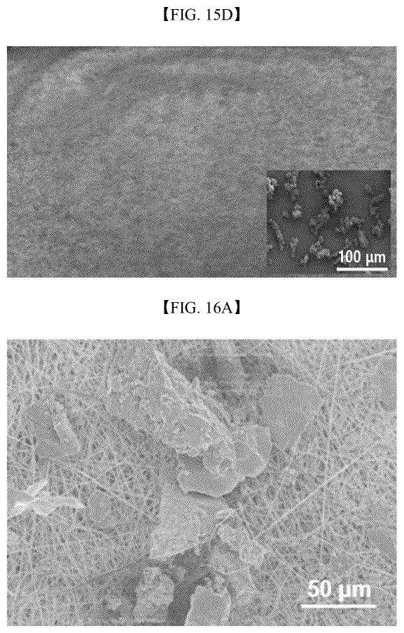

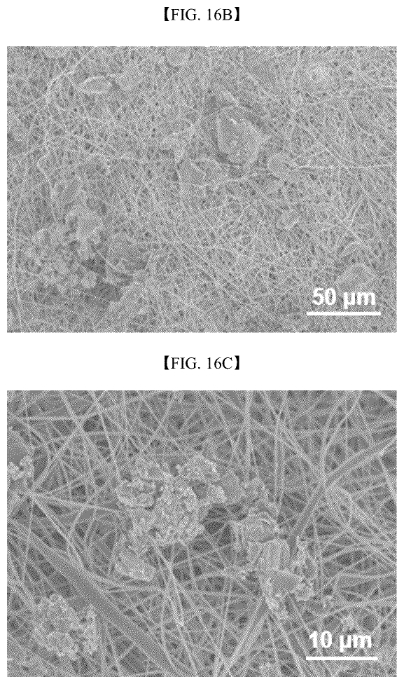

FIGS. 15A to 15D illustrate optical microscope images of contaminant particles of various sizes, the inset in each image is an SEM image for confirming the average diameter of the contaminant particles, and FIGS. 16A to 16D illustrate SEM images for observing the adhesion pattern of contaminant particles of various sizes on the PCL mesh.

FIGS. 15A to 15D respectively show the distribution of particles with sizes of about 300 μm, 125 μm, 70 μm, and 20 μm. FIGS. 16A to 16D show the state of particles with sizes of 300 μm or less, 125 μm or less, 70 μm or less, and 20 μm or less, respectively, attached to the mesh surface.

As shown in FIGS. 15A to 15D and FIGS. 16A to 16D, particles (e.g., 300 μm, 125 μm, 70 μm) larger than the pore size were observed to be placed on the surface without passing through the pores of the mesh. On the other hand, in the case of particles similar to or smaller than the pore size (e.g., 20 μm), a distinct pattern of adhesion to the PCL mesh fiber surface was observed due to electrostatic attraction, inter-fiber capillary effects, and a high specific surface area.

Therefore, the PCL mesh of the present disclosure performs a dual protective function, exerting both a physical blocking effect and an electrostatic trapping effect depending on the particle size, and it can be confirmed that it prevents direct contact with large foreign substances that may occur during external exposure of a microneedle part, while also effectively suppressing the adhesion of small fine particles, thereby securing both hygienic stability and formulation stability upon skin application.

In addition, it can be confirmed that the E-MAP structure of the present disclosure goes beyond simply protecting the drug coating layer and serves as a structural barrier capable of blocking external contaminants.

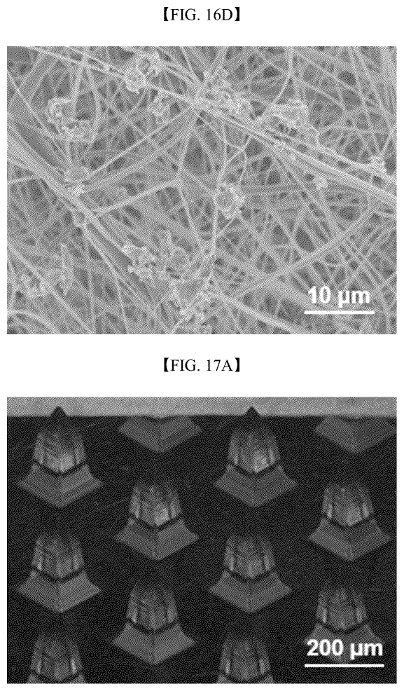

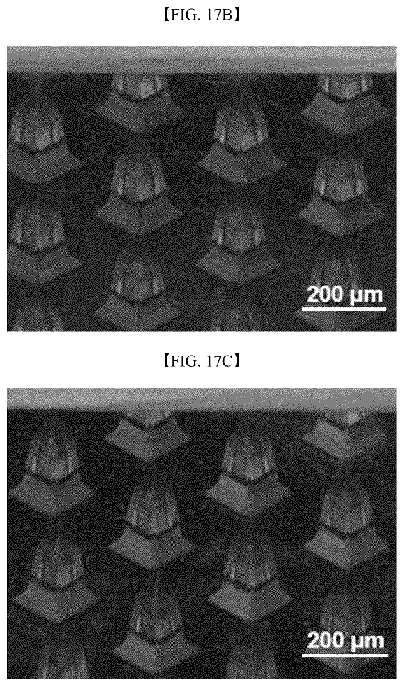

FIGS. 17A to 17D illustrates the surface images of microneedle parts observed with an optical microscope after removing the PCL mesh following the induction of penetration by contaminants of various sizes.

FIGS. 17A to 17D respectively show the cases using particles with an average diameter of about 300 μm or less, about 125 μm or less, about 70 μm or less, and about 20 μm or less.

As shown in FIGS. 17A to 17D, no traces of particles penetrating or remaining on the surface of the microneedle part were observed under all particle size conditions. The results of FIGS. 17A to 17D means that the PCL mesh not only provides a simple mechanical block for contaminants larger than the pores, but also effectively suppresses particles from reaching the coating layer on the surface of the microneedle part by inducing even fine particles to be adsorbed or attached to the mesh surface itself, and the contaminant-blocking effect is determined to be due to the porous structure, high surface area, and electrostatic interaction of the PCL mesh.

Therefore, through the results of FIGS. 17A to 17D, it can be seen that the PCL mesh according to the present disclosure acts as a multi-layered protective film for a microneedle part-coating layer.

In other words, since it is confirmed that it can stably protect a microneedle part-coating layer from contaminant particles of various sizes that may occur in the actual use environment by performing a physical blocking function for large-sized particles and inducing adsorption and trapping for small-sized particles, it can be seen that the PCL mesh of the present disclosure is a key structural element that guarantees the hygienic stability and reliability upon in-vivo application of a microneedle part-based drug delivery system.

### [Experimental Example 9] Evaluation of Drug Delivery Performance of Coated Microneedle Part Protected with PCL Mesh

### 9-1. Evaluation of Permeation Performance of E-C-MAP

### (1) In-Vitro Insertion Experiment

A microneedle part coated with 2 w/v % trypan blue was vertically attached to the previously fabricated PDMS model, maintained for about 1 second with a force of 5 kg, and then removed. Next, the PDMS surface and the microneedle part were observed using an optical microscope, and the insertion marks and any damage to the microneedle part were checked.

### (2) Ex Vivo Insertion Experiment

Anex vivo experiment was conducted to evaluate the skin permeation ability of E-C-MAP. The pig skin (Cronex) used in the experiment was full-thickness skin with a thickness of 1 mm, and the size of the skin specimen was 2.5 cm×2.5 cm. It was stored frozen before the experiment and thawed at room temperature immediately before the experiment for use.

A microneedle part coated with 2 w/v % trypan blue was attached to pig skin with a load of about 5 kg for 10 seconds and then removed, after which the skin surface was observed using an optical microscope. Next, the number of stained holes formed in the microneedle part was counted, and the permeation rate of E-C-MAP was analyzed by comparing it with that of 0 μm-MAP (bare needles), C-MAP, and the total number of microneedle parts.

A microneedle part coated with 2 w/v % FITC was attached to pig skin with a load of about 5 kg for 10 seconds and then removed, after which the shape of the microneedle part and a polymer mesh was observed using an optical microscope. Next, the pig skin to which the microneedle part was applied was observed using a confocal laser scanning microscope (Nikon Eclipse C1si), and whether the coating layer permeated into the skin was confirmed through fluorescence imaging.

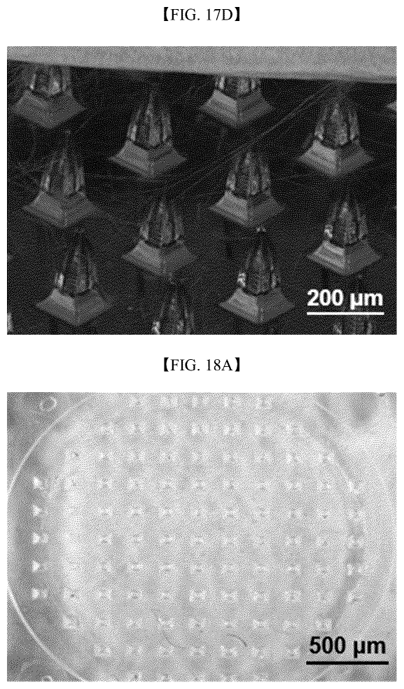

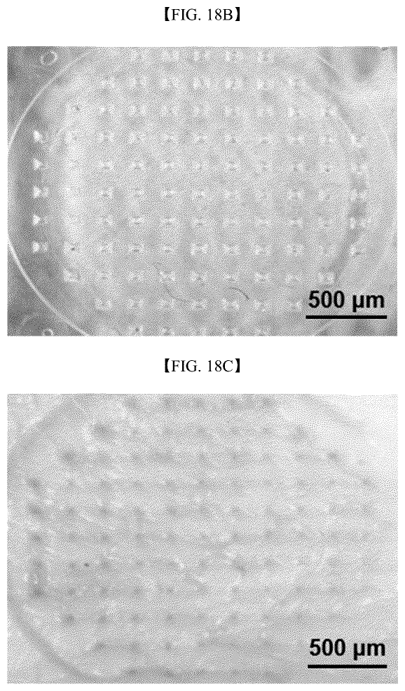

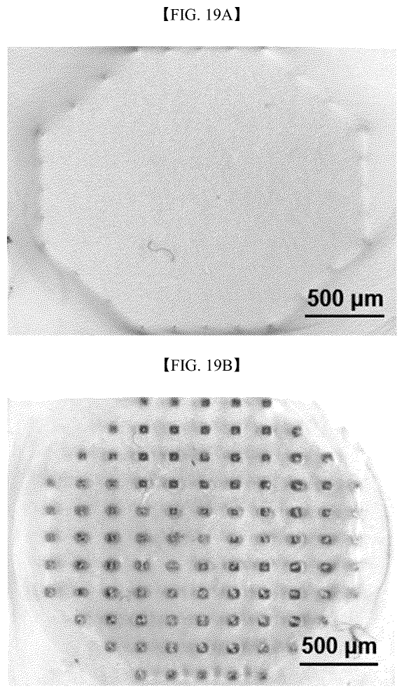

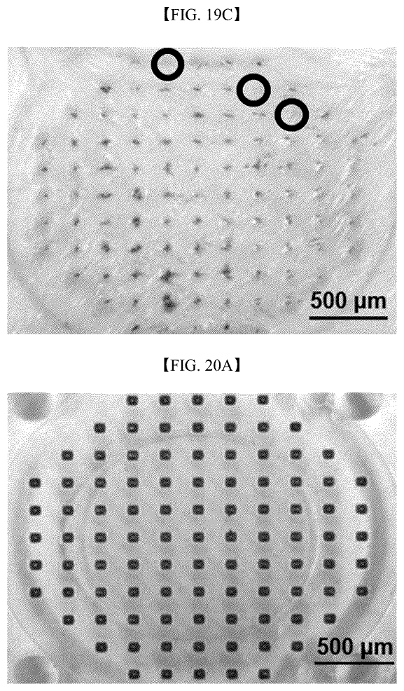

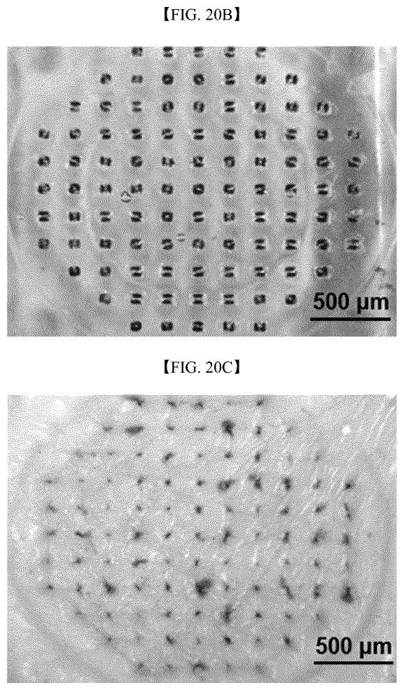

FIGS. 18A to 18C through FIGS. 20A to 20C illustrate the optical microscope images of bare needles (FIGS. 18A to 18C), E-C-MAP (FIGS. 19A to 19C), and C-MAP (FIGS. 20A to 20C) before and after permeating PDMS and pig skin.

In FIGS. 18A to 18C through FIGS. 20A to 20C, A shows images before PDMS permeation of bare needles (FIGS. 18A to 18C), E-C-MAP (FIGS. 19A to 19C), and C-MAP (FIGS. 20A to 20C), respectively; B shows images after PDMS permeation of bare needles (FIGS. 18A to 18C), E-C-MAP (FIGS. 19A to 19C), and C-MAP (FIGS. 20A to 20C); and C shows images after pig skin permeation of bare needles (FIGS. 18A to 18C), E-C-MAP (FIGS. 19A to 19C), and C-MAP (FIGS. 20A to 20C).

As shown in FIGS. 18A to 18C through FIG. 20A to 20C, the bare needles and C-MAP not only completely permeated PDMS, but were also confirmed to be fully inserted without damage when applied to pig skin.

However, in the case of E-C-MAP, it was observed that some needles were not inserted during the skin penetration process, and the insertion rate was somewhat reduced compared to the bare needles and C-MAP. However, even in the case of E-C-MAP, the insertion rate is confirmed to be at a high level of 96.9%.

According to the results of FIGS. 18A to 20C, it is confirmed that even when a PCL mesh is applied, the mechanical stiffness and structural characteristics of the microneedle part are maintained, allowing it to effectively penetrate simulated tissues such as skin and PDMS.

In addition, while the PCL mesh may cause a slight decrease in insertion depth during the insertion process, it does not significantly affect the overall insertion function, and it can be confirmed that insertion occurs while maintaining the arrangement stability of a microneedle part.

Therefore, it can be confirmed that the microneedle part system with the PCL mesh protective layer of the present disclosure stably maintains its insertion rate and skin penetration function even with the protective layer formed, and it is analyzed that the application of the PCL mesh protective layer has a limited effect on insertability while providing a formulation protection function.

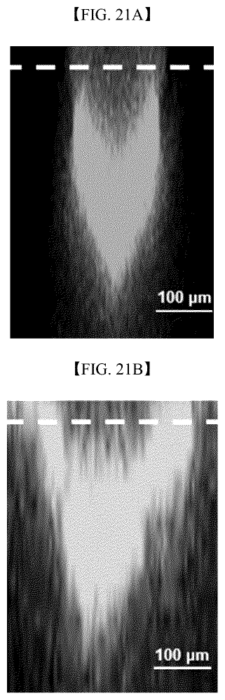

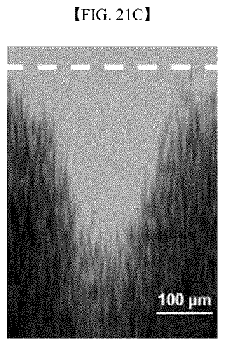

FIGS. 21A to 21C illustrate the confocal microscope images of the microneedle part before and after insertion into pig skin. The dashed line at the top of the figure indicates the surface of the pig skin, and the area below the dashed line corresponds to the inside of the skin.

FIG. 21A shows the state of the C-MAP before skin insertion, and no clear fluorescent signal is observed below the dashed line, showing that the coating layer material was not delivered into the skin. However, some scattered light is observed near the surface, which is interpreted as a surface residual signal rather than delivery due to actual insertion.

FIG. 21B is an image after inserting the C-MAP into the skin, and it is confirmed that the yellow fluorescent shape continuously extends to the area inside the skin below the dashed line. According to FIG. 21B, it can be seen that as the microneedle part mechanically penetrates the skin barrier, the coating layer material is directly delivered into the skin.

FIG. 21C is a result after inserting the E-C-MAP into the skin, and although a fluorescent signal is observed below the dashed line, it can be seen that the depth of the signal is relatively shallow and its distribution is locally limited.

The result of FIG. 21C is determined to be a consequence of the insertion depth of the microneedle part being somewhat suppressed as the PCL mesh acts as a protective film, or the delivery of the coating layer material being only partially achieved.

Therefore, according to the result of FIGS. 21A to 21C, it can be confirmed that the coating layer material is effectively delivered into the skin by the insertion of the microneedle part, and it can be seen that even when the PCL mesh protective layer of the present disclosure is applied, insertion is possible, but the insertion depth and delivery efficiency are reduced to some extent.

However, since insertion is possible and drug delivery is maintained even when the PCL mesh protective layer is applied, it can be confirmed that the introduction of the PCL mesh protective layer provides both a protective function and insertability at the same time.

### 9.2 E-C-MAP Delivery Efficiency Analysis

A microneedle part coated with FITC (C-MAP) and a microneedle part with a protective layer formed on top of the FITC-coated microneedle part by an electrospinning method (E-C-MAP) were insertedex vivo into pig skin, left for 5 minutes at 37° C., and then removed from the skin.

Next, the administration site was wiped with a wet cotton swab, and the removed microneedles were placed in 1 mL of distilled water and treated for 10 minutes to recover the FITC remaining on the surface. The used cotton swab was also treated in the same manner in a separate 1 mL of distilled water to recover the residual FITC.

The fluorescence intensity of the recovered samples was measured using a microplate reader, and the amount of FITC delivered to the skin was calculated by subtracting the residual amount from the initial loading amount, with the results shown in Table 4 below.

TABLE 4

| | C-MAP | E-C-MAP |
|---|---|---|
| Delivered amount (μg) | 365.29 ± 6.97 | 408.82 ± 94.06 |
| Delivery efficiency (%) | 47.65 ± 0.91 | 53.33 ± 12.27 |

As shown in Table 4, the delivered amount of C-MAP was measured to be about 365.29±6.97 μg and its delivery efficiency was about 47.65±0.91%, while the delivered amount of E-C-MAP was about 408.82±94.06 μg and its delivery efficiency was about 53.33±12.27%, confirming that even when the PCL mesh protective layer was applied, the skin delivery efficiency of the microneedle part was at a similar level or rather improved to some extent.

The results of Table 4 are interpreted to mean that the protective layer improves drug delivery efficiency to some extent by suppressing damage or detachment of the coating layer during the insertion process.

However, compared to C-MAP, E-C-MAP shows a somewhat larger standard deviation in delivery efficiency, which indicates that variations in the thickness, uniformity, and insertion depth of the protective layer structure affect the results.

Therefore, the results of Table 4 show that while the PCL mesh protective layer can enhance coating stability and maintain delivery efficiency in a skin-insertable drug delivery system, it is necessary to secure the uniformity of results through manufacturing process optimization.

In the present disclosure, by introducing a fiber layer, formed by electrospinning a lipophilic polymer on the top of microneedles, as a protective layer, the microneedles can be protected from contaminants such as moisture and pollutants during long-term storage and exposure situations of the microneedles.

In addition, in the present disclosure, to prevent the problem of decreased skin insertability of the microneedles due to the protective layer, an electrospun fiber layer is designed such that tips of a microneedle part can penetrate the protective layer or be inserted and pass through pores with a mesh structure, thereby securing skin permeability and drug delivery efficiency while maintaining a protective function.

Furthermore, in the present disclosure, insertability can be maintained due to the porous mesh structure, low moisture permeability can be achieved, and stability can be secured by adding a hygroscopic material; and due to the process characteristic of being able to form a protective layer via an electrospinning process regardless of the microneedle formulation, the electrospun fiber layer can be easily applied to various microneedles such as coated, solid, and dissolvable formulations.

Although the present disclosure has been described through limited examples and drawings, the present disclosure is not intended to be limited to the examples. Those skilled in the art will appreciate that various modifications, additions and substitutions are possible, without departing from the scope and spirit of the disclosure. Therefore, the scope of the present disclosure should not be limited to the described examples, but should be defined not only by the claims described below but also by equivalents of these claims.

[Description of Symbols]

| | |
|---|---|
| 100 microneedle patch | 110 base |
| 120 needle part | 130 microneedle part |
| 140 polymer fiber layer | 210 active pharmaceutical ingredient |

What is claimed is:

1. A microneedle patch, comprising:
a microneedle part comprising a base and a plurality of needles for forming tips protruding in a sharp shape from one surface of the base; and
a polymer fiber layer formed to cover the microneedle part to perform a function of protecting an active pharmaceutical ingredient contained in the needles from moisture and contaminants,
wherein an edge of the polymer fiber layer is connected to an edge of one surface of the base, and a central part of the polymer fiber layer is supported in a state of being stacked and placed on tips of the needles.

2. The microneedle patch according to claim 1, wherein the needles are made of at least one of a solid formulation, a dissolvable formulation, a powder-attached formulation, and a coated formulation, and
the tips of the needles pass through the polymer fiber layer and are inserted into skin when the microneedle patch is used.

3. The microneedle patch according to claim 1, wherein the polymer fiber layer comprises a lipophilic and biocompatible polymer, and forms a stacked structure in a form of a porous mesh or film comprising pores.

4. The microneedle patch according to claim 3, wherein the polymer fiber layer further comprises a hygroscopic material to suppress denaturation of the active pharmaceutical ingredient and denaturation of the needles due to moisture absorption during long-term storage.

5. The microneedle patch according to claim 3, wherein the polymer fiber layer increases hydrophobicity by a porous fiber structure comprising the pores and by surface roughness to suppress penetration of external moisture.

6. The microneedle patch according to claim 3, wherein the polymer fiber layer controls a size of the pores and an electrostatic interaction between the fibers and the contaminants to suppress the contaminants from reaching a surface of the microneedle part.

7. The microneedle patch according to claim 3, wherein the tips of the needles pass through the pores, formed in the mesh-form polymer fiber layer, and are inserted into skin, when the microneedle part is used.

8. The microneedle patch according to claim 3, wherein the tips of the needles pass through the film-form polymer fiber layer by locally fracturing the film-form polymer fiber layer due to high local stress acting on the film-form polymer fiber layer and are inserted into skin, when the microneedle part is used.

9. The microneedle patch according to claim 1, wherein the polymer fiber layer has a thickness of 10 to 50 μm.

10. The microneedle patch according to claim 1, wherein the polymer fiber layer has a pore size of 0.5 to 4 μm.

11. A method of manufacturing a microneedle patch, the method comprising:
forming a microneedle part comprising a base and a plurality of needles for forming tips protruding in a sharp shape from one surface of the base; and
forming a polymer fiber layer formed to cover the microneedle part by electrospinning a lipophilic and biocompatible polymer solution onto the microneedle part to perform a function of protecting an active pharmaceutical ingredient contained in the needles from moisture and contaminants,
wherein an edge of the polymer fiber layer is connected to an edge of one surface of the base, and a central part of the polymer fiber layer is supported in a state of being stacked and placed on tips of the needles.

12. The method according to claim 11, wherein in the forming of the polymer fiber layer, the polymer solution comprises polycaprolactone (PCL).

13. The method according to claim 11, wherein in the forming of the polymer fiber layer, the polymer solution comprises a cosolvent to ensure shape stability and fiber uniformity of the polymer fiber layer.

14. The method according to claim 13, wherein the cosolvent comprises acetone and acetic acid, and
a composition ratio of the acetone to the acetic acid is 7:3 to 9:1.

15. The method according to claim 11, wherein in the forming of the polymer fiber layer, the polymer solution is electrospun at an electrospinning speed of 8 to 10 mL/h.
